(19) United States
(12) Patent Application Publication
(10) Pub. No.: US 2022/0297126 A1
(43) Pub. Date: Sep. 22, 2022

(54) Microfluidic Pipette Aspirators for Large-Scale Analysis of Single Cells, Clusters and Their Sub-Populations

(71) Applicant: Texas Tech University System, Lubbock, TX (US)

(72) Inventors: Siva A. Vanapalli, Lubbock, TX (US); Nabiollah Kamyabi, Lubbock, TX (US); Adity Pore, Lubbock, TX (US); Shamim Ahmed, Lubbock, TX (US); Swastika S. Bithi, Lubbock, TX (US)

(21) Appl. No.: 17/630,975

(22) PCT Filed: Jul. 28, 2020
(86) PCT No.: PCT/US2020/043808

§ 371 Date: Jan. 28, 2022

Related U.S. Application Data
(60) Provisional application No. 62/880,161, filed on Jul. 30, 2019.

(51) Int. Cl. B01L 3/00; G01N 33/50

(52) U.S. Cl. CPC B01L 3/502761; G01N 33/5011; B01L 2200/0668; B01L 2300/0636; B01L 2300/0829; B01L 2400/049; B01L 2300/0864

(57) ABSTRACT

The present invention includes a device and a method of using the device, wherein the device is a microfluidic device for single or multicell capture comprising: a substrate; one or more microgrooves or microtubes disposed within the substrate, each N microgroove or microtube having a first end and a second end, wherein a width of the microgroove or microtube is a diameter of a target cell or a group of cells, wherein the microgroove or microtube comprises one or more chambers; a fluid input disposed within the substrate in fluid communication with the first end of the one or more microgrooves or microtube; and a fluid output disposed within the substrate in fluid communication with the second end of the one or more microgrooves or microtube, or the one or more chambers, wherein one or more cells that are captured in the microgroove can be analyzed as a single cell.

### CROSS-REFERENCE TO RELATED APPLICATIONS

None.

### TECHNICAL FIELD OF THE INVENTION

The present invention relates in general to the field of microfluidics, and to a novel device and method for using Microfluidic pipette aspirators (μFPA) for capture of single cells and groups of cells at large-scale for characterizing their mechanical properties and lineage-based analysis of drug response which is relevant for applications in biology and medicine.

### STATEMENT OF FEDERALLY FUNDED RESEARCH

None.

### BACKGROUND OF THE INVENTION

Without limiting the scope of the invention, its background is described in connection with cell analysis and microfluidic devices.

One such patent is U.S. Pat. No. 6,653,124, entitled, “Array-based microenviroment for cell culturing, cell monitoring and drug-target validation”, issued to Freeman, and is said to teach a microfluidic device that allows cell culturing and drug validation. The device is said to allow cell culturing in an array format so that cells can be directly placed in the bottom of the plate. The microchambers in this lab-on-a-chip device are supplied with media by multiple channels, allowing the user the choice of using either homogenous or heterogenous assays by implementing fluorescently labeled molecules, nucleic acids, or fluorescent tags based on antibodies.

Another patent is U.S. Pat. No. 8,748,180, entitled, “Microfluidic device for pharmacokinetic-pharmacodynamic study of drugs and uses thereof”, issued to Shuler and Sung, and is said to teach a microfluidic device for pharmacokinetic and pharmacodynamic studies is shown. The device can culture cells and is assembled by a base layer, cell culture chambers of one or more cells. The layer with the cell culture chambers is positioned between the fluidic channel and the base layer so that the cell culture chambers can be fluidically connected and produce flow rates.

U.S. Patent Application No. US20160167051A1, entitled, “Microfluidic devices and methods for cell processing”, filed by Collins, is said to teach a microfluidic cell sorter that allows cell sorting by using flow-based field potential sensing and sorting. The spiral device has three functions; it can sort stem cells, circulating tumor cells, and cell culturing. The stem cells can be sorted by using electrodes for impedance sensing, stimulus current and discrete recording of the time domain stimulus by using an array of electrodes of 20 pairs or more in the path were cells flow. Circulating tumor cells can also be sorted by using the spiral microfluidic chambers. These spiral microfluidic chambers are periodically interconnected and are named yoked channels. In these channels the cells are delivered through the inlet and then flowed through a primary spiral microfluidic channel and secondary microfluidic channel where they are separated.

An article by V. Lecault et al., entitled, “High-throughput analysis of single hematopoietic stem cell proliferation in microfluidic cell culture arrays”, Nature Methods volume 8, pages 581-586 (2011), is said to teach a microfluidic platform containing thousands of nanoliter-scale chambers suitable for live-cell imaging studies of clonal cultures of nonadherent cells with precise control of the conditions, capabilities for in situ immunostaining and recovery of viable cells. If further said to teach that the platform mimics conventional cultures in reproducing the responses of various types of primitive mouse hematopoietic cells with retention of their functional properties, as demonstrated by subsequent in vitro and in vivo (transplantation) assays of recovered cells.

An article by Somasherak, et al., entitled “Tracking cancer cell proliferation on a CMOS capacitance sensor chip”, Biosensors and Bioelectronics, Volume 23, Issue 10, 15 May 2008, Pages 1449-1457 is said to teach a device that can track cancer cell proliferation by using capacitance sensors for monitoring their growth. The miniaturized device uses complementary metal-oxide semiconductor (CMOS) sensors to measure the capacitance coupling between on-chip sensing electrodes and the cellular matrix that is cultured on them to track proliferation. Within the growth chamber, the electrodes are arranged in a planar configuration and are insulated from the cell environment.

Finally, an article by S. Faley, et al., entitled, “Microfluidic single cell arrays to interrogate signaling dynamics of individual, patient-derived hematopoietic stem cells”, Lab on a Chip, Issue 18, 2009, is said to teach a platform to interrogate hundreds of non-adherent cells. The device is said to be designed by aiming to solve the problem of cell isolation in hematopoietic stem cell disorders, since these cells are difficult to isolate.

### SUMMARY OF THE INVENTION

In one embodiment, the present invention includes a microfluidic device for single or multicell capture comprising: a substrate; one or more microgrooves or microtubes disposed within the substrate, each microgroove or microtube having a first end and a second end, wherein a width of the microgroove or microtube is a diameter of a target cell or a group of cells, wherein the microgroove or microtube comprises one or more chambers, wherein the microgroove or microtube has a constricted end and the microtube or microtube has a micropillar that bifurcates a fluid flow to capture a target cell or a groups of cells; a fluid input disposed within the substrate in fluid communication with the first end of the one or more microgrooves or microtube; and a fluid output disposed within the substrate in fluid communication with the second end of the one or more microgrooves or microtube, or the one or more chambers, wherein one or more cells that are captured in the microgroove can be analyzed as a single cell. In one aspect, the diameter of the target cell is between 1 to 100 μm in diameter. In another aspect, the one or more chambers are rectangular, circular, or triangular shaped. In another aspect, the micropillar in the microtube is circular, rectangular or triangular shaped. In another aspect, the target cell is a cancer cell. In another aspect, the microfluidic device, further comprises an imaging device, wherein the imaging device detects one or more cells in the microgroove. In another aspect, the substrate is biocompatible or is a material coated to be biocompatible. In another aspect, the substrate is selected from at least one of glass, silicon, polymer, plastic, metal, ceramic, semiconductor, or any combination thereof. In another aspect, a length of each microgroove or microtube is between about 1 to 75 μm and a height depth of the microgroove is between about 1 to 100 μm. In another aspect, the target cell is a mammalian, plant, insect, or bacterial cell. In another aspect, the microgrooves or microtubes further comprise a portion that narrows to a size that is less than a diameter of a target cell to trap the target cell. In another aspect, the microgrooves or microtubes are on a first plane, and at least one of the input or output are on a second plane. In another aspect, the microgrooves or microtubes are on a first plane, and at least one of the input or output are on a second plane, wherein the input, the output, or both are above or below the first plane. In another aspect, a fluid drives the target cells or group of cells to enter the microgrooves or microtubes toward the fluid output. In another aspect, a fluid pressure drop is equally distributed among the microgrooves or microtubes. In another aspect, the microgrooves or microtubes are sized to hold one or more target cells. In another aspect, a pillar gap in the microtubes is adjusted to capture cells of a larger size and let through smaller cells, to enable size-selective sorting of a mixed cellular population. In another aspect, the microgrooves or microtubes can hold one or more daughter cells from target cells. In another aspect, the input comprises a first buffer exchange/feeding port or fluid reservoir. In another aspect, the output port comprises a reservoir or a site for aspiration of a fluid in the device.

In another embodiment, the present invention includes a method of making a microfluidic device having one or more microgrooves comprising: providing a substrate; forming one or more microgrooves or microtubes in or on the substrate, wherein each microgroove or microtube has a first end and a second end, wherein a width of the microgroove is a diameter of a target cell; connecting a fluid input to the first end of the one or more microgrooves or microtubes; and connecting a fluid output to the second end of the one or more microgrooves or microtubes, wherein one or more cells that are captured in the microgroove or microtubes can be analyzed as a single cell. In one aspect, the diameter of the target cell is between 1 to 100 μm in diameter. In another aspect, the target cell is a cancer cell. In another aspect, the method further comprises using an imaging device to capture an image, wherein the imaging device detects one or more cells in the microgroove. In another aspect, the substrate is biocompatible or is a material coated to be biocompatible. In another aspect, the substrate is selected from at least one of glass, silicon, polymer, plastic, metal, ceramic, semiconductor, or any combination thereof. In another aspect, a length of each microgroove or microtube is between about 1 to 75 μm and a height depth of the microgroove is between about 1 to 100 μm. In another aspect, the target cell is a mammalian, plant, insect, or bacterial cell. In another aspect, the microgrooves or microtubes further comprise a portion that narrows to a size that is less than a diameter of a target cell to trap the target cell. In another aspect, the microgrooves or microtubes are on a first plane, and at least one of the input or output are on a second plane. In another aspect, the microgrooves or microtubes are on a first plane, and at least one of the input or output are on a second plane, wherein the input, the output, or both are above or below the first plane. In another aspect, the input comprises a first buffer exchange/feeding port or fluid reservoir. In another aspect, the output port comprises a reservoir or a site for aspiration of a fluid in the device.

A method of measuring cellular mechanical strength using a microfluidic device having one or more microgrooves comprising: providing a microfluidic device for single or multicell capture comprising: a substrate; one or more microgrooves or microtubes disposed within the substrate, each microgroove or microtube having a first end and a second end, wherein a width of the microgroove or microtube is a diameter of a target cell or a group of cells, wherein the microgroove or microtube comprises one or more chambers; a fluid input disposed within the substrate in fluid communication with the first end of the one or more microgrooves or microtubes; a fluid output disposed within the substrate in fluid communication with the second end of the one or more microgrooves or microtubes, or the one or more chambers, wherein one or more cells that are captured in the microgroove can be analyzed as a single cell; and directing an imaging device to the one or more cells, such that the imaging device detects one or more cells in the microgroove or microtube; imaging the one or more cells; treating the one or more cells with one or more active agents; and determining the effect of the one or more active agents on the one or more cells by detecting changes to the one or more cells in the microgrooves or microtubes. In one aspect, the diameter of the target cell is between 1 to 100 μm in diameter. In another aspect, the target cell is a cancer cell, and the one or more active agents is an anti-neoplastic agent. In another aspect, the one or more chambers are rectangular, circular, or triangular shaped. In another aspect, the method further comprises an imaging device, wherein the imaging device detects one or more cells in the microgroove or microtube. In another aspect, the substrate is biocompatible or is a material coated to be biocompatible. In another aspect, the substrate is selected from at least one of glass, silicon, polymer, plastic, metal, ceramic, semiconductor, or any combination thereof. In another aspect, a length of each microgroove or microtube is between about 1 to 75 μm and a height depth of the microgroove is between about 1 to 100 μm. In another aspect, the target cell is a mammalian, plant, insect, or bacterial cell. In another aspect, the microgrooves or microtubes further comprise a portion that narrows to a size that is less than a diameter of a target cell to trap the target cell. In another aspect, the microgrooves or microtubes are on a first plane, and at least one of the input or output are on a second plane. In another aspect, the microgrooves or microtubes are on a first plane, and at least one of the input or output are on a second plane, wherein the input, the output, or both are above or below the first plane. In another aspect, the input comprises a first buffer exchange/feeding the input port or a fluid reservoir. In another aspect, the output port comprises a reservoir or a site for aspiration of a fluid in the device. In another aspect, a fluid drives the target cells or group of cells to enter the microgrooves or microtubes toward the fluid output. In another aspect, a fluid pressure drop is equally distributed among the microgrooves or microtubes. In another aspect, the microgrooves are sized to hold one or more target cells. In another aspect, the microgrooves or microtubes can hold one or more daughter cells from target cells. In another aspect, the captured cells or groups of cells can be removed by reversing a flow, and replenished with new cells for further analysis. In another aspect, the method further comprises capturing a cell or groups of cells, and adding reagents to stain specific components in the cell.

### BRIEF DESCRIPTION OF THE DRAWINGS

For a more complete understanding of the features and advantages of the present invention, reference is now made to the detailed description of the invention along with the accompanying figures and in which:

FIGS. 1A and 1B show a summary of the overall method and a detailed structure for use with the present invention. FIG. 1A is a Schematic of a standard drug assay and fate of the cancer cell after chemotherapy. FIG. 1B shows MCF-7 cells identified as apoptotic, quiescent and proliferating after 48-hour drug exposure.

FIGS. 2A to 2D show a microfluidic setup for single cell isolation. FIG. 2A is a schematic of the experimental setup showing the reservoir filled with cells and a 200 μL pipettor used for aspiration. FIG. 2B is an image of the microfluidic device alongside a penny, showing the microfluidic channels and sample reservoir (filled with dye). FIG. 2C is a scanning electron microscope image of the microfluidic device used for single cell isolation. FIG. 2D shows cells within the microfluidic device used for single cell isolation.

FIGS. 3A to 3C show the optimization of cell concentration for one embodiment of the present invention. FIG. 3A shows the trapping efficiencies at different cell concentrations. Choosing the optimum concentration of 2000 cells/100 μL for maximum trapping efficiency (1600 traps in the microfluidic device). FIG. 3B shows MCF-7 cell trapping at different concentrations. The concentrations are in 100 μL of media. FIG. 3C shows the full occupancy distribution of different number of cells in the microfluidic device. The average number of cells and standard deviation were calculated from 3 replicates.

FIGS. 4A and 4B show the proficiency of single cell proliferation. FIG. 4A shows the proliferation capacity of MCF-7 cells over a period of 48 hours after isolation. FIG. 4B shows the single MCF-7 cells at time t=0 hr and proliferated MCF-7 cells stained with DAPI after time t=24 hr and t=48 hr. The average number of cells and standard deviation were calculated from 3 replicates. Scale bar is 100 μm.

FIGS. 5A and 5B show the drug susceptibility of single cells. FIG. 5A shows a dose response curve with doxorubicin and MCF-7, MB-231 after 24 hours of incubation. FIG. 5B shows proliferation capability of drug resistant MB-231 and MCF-7 cells in the presence of 0.01 μM, 0.001 μM and 0.0001 μM concentrations of Dox.

FIGS. 6A and 6B are isometric views of one design of the device of the present invention. FIG. 6A shows the formula and shape of the device, with FIG. 6B showing a close-up view of the microgroove/microtube for isolating single cells.

FIG. 7 shows different views of another design of the device of the present invention. FIG. 7 includes an isometric view that includes the source of liquid, the array of cells, and the vacuum or outlet for the liquid, shows a close-up view of the microgroove/microtube for isolating single cells that include chambers that help drive cells into the microgrooves/microtubes, shows the relative size of the device when compared to a penny, and a broader view showing the arrays and aspirators for drawing the cells into the microgrooves/microtubes.

FIGS. 8A to 8D are a side view with the inflow and outflow of the device. FIG. 8A shows the locations for pressure and other measurements from simulations of the device.

FIG. 8B is a graph that shows the pressure at the various trap numbers. FIG. 8C is a graph that shows the change in pressure versus channel of aspiration. FIG. 8D is a graph that shows the change in pressure and the array number.

FIGS. 9A and 9B are graphs that show rheological cell-model selection simulations. FIG. 9A is a graph that shows the length versus time. FIG. 9B is a graph that shows the change in pressure versus length.

FIGS. 10A to 10C show the mechanical characterization of cells in the device of the present invention. FIG. 10A shows a top-view of cells in the microgrooves/microtubes of the present invention. FIG. 10B is a graph that shows the frequency of cells versus the Young's modulus of the cells. FIG. 10C is a graph that shows the Young's modulus of the cells versus the cell diameters.

FIG. 11 is a graph that shows the Young's modulus of MB231 cells, when cells are untreated, in 10% BSA, and 5% pluronic.

FIGS. 12A and 12B show the sensitivity of different cells (MB231 and MCF7) to different drugs (blebbistatin and paclitaxel), and the Young's modulus of the cells. FIG. 12A is a graph that shows the Young's modulus of MB231 cells under control conditions, treated with blebbistatin or paclitaxel. FIG. 12B is a graph that shows the Young's modulus of MCF7 cells showing whether they are in G1, S, or G2 phase.

FIG. 13 is a graph that shows the Young's modulus of CTC-derived cell lines: MB231, BRX68, BRX07, and LM1.

### DETAILED DESCRIPTION OF THE INVENTION

While the making and using of various embodiments of the present invention are discussed in detail below, it should be appreciated that the present invention provides many applicable inventive concepts that can be embodied in a wide variety of specific contexts. The specific embodiments discussed herein are merely illustrative of specific ways to make and use the invention and do not delimit the scope of the invention.

To facilitate the understanding of this invention, a number of terms are defined below. Terms defined herein have meanings as commonly understood by a person of ordinary skill in the areas relevant to the present invention. Terms such as “a”, “an” and “the” are not intended to refer to only a singular entity, but include the general class of which a specific example may be used for illustration. The terminology herein is used to describe specific embodiments of the invention, but their usage does not limit the invention, except as outlined in the claims.

Drug resistance is a highly recognized hallmark of cancer. Drug resistance can preexist in patients prior to chemotherapy or it can arise post drug treatment.¹⁻⁶ These drug resistant cells can be lethal to the patient as they can aid in the recurrence or relapse of cancer.⁷ Chemotherapy is known to have adverse side effects on the health of patients. Owing to the predominating drug resistance, administering a higher concentration of drug and having the same response as that of a lower concentration can have more side effects on patient.^{8,9} Thus, tumorigenic drug resistances play a major role in impeding contemporary cancer therapies.

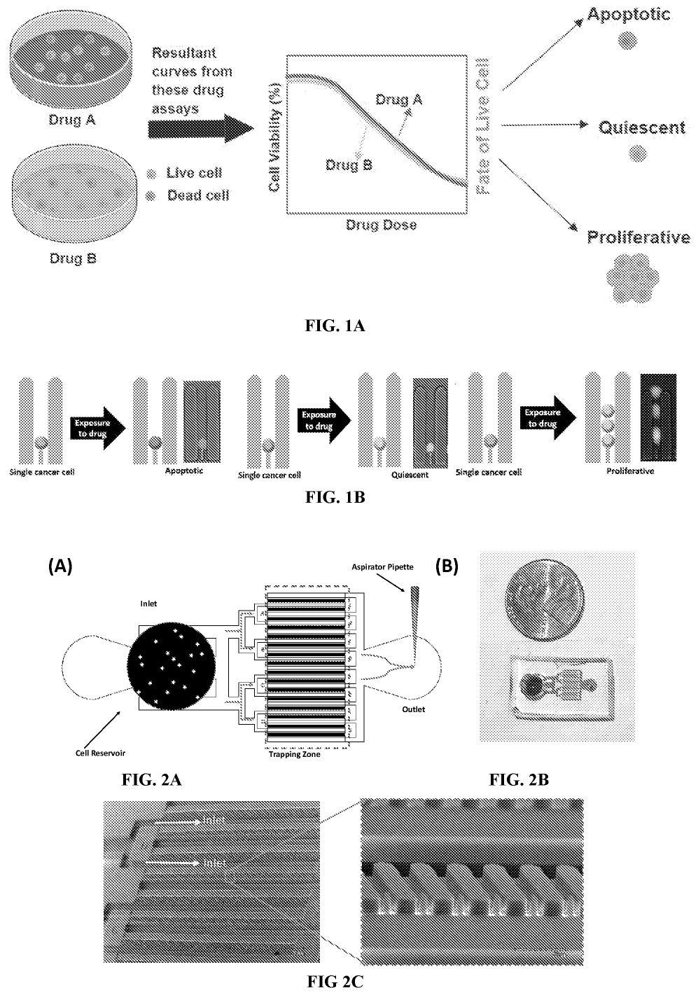

Drug resistance arising due to chemotherapy can be classified as intrinsic and acquired. Intrinsic drug resistance is inherently present in some cancer cells before undergoing chemotherapy; whereas acquired drug resistance is developed in cancer cells after undergoing mutations in the presence of chemotherapeutic reagents.^{1,4,5,10} The cellular fates of these drug resistant cells can be classified as proliferative and non-proliferative; the non-proliferative state can further be classified as quiescent and senescent.¹¹⁻¹³ Quiescence and senescence have often been used indistinguishably.¹¹ Quiescence has been referred to as a reversible non-proliferative state^{11,14}, whereas senescence is defined as a lack of proliferative potential resulting into cell death.^{11,15} Thus, there is a need for approaches that can give information about these underlying states of drug resistance, where a proliferative or quiescent cell can prove to be a tipping point in the treatment of a patient. FIG. 1A shows the classification of these drug resistant cells for a comparative drug study.

Several in vitro chemosensitivity assays such as clonogenic assay¹⁹, FMCA assay²⁰, collagen gel droplet drug sensitivity test²¹, KERN assay²², flow cytometry assay²³, ATP viability assay²⁴, MTT assay²⁴ etc. have been developed to identify drug resistance.²⁵ These bulk assays identify a drug resistant population as those that survived after exposure to a certain dose of drugs. For example, as shown in FIG. 1A, drug A and drug B when evaluated using bulk assays can show similar viability curves, but the fate of individual survivor cells can be either quiescent or proliferative. Thus, single cell analysis can provide an insight on the heterogeneity of these drug resistant cells.

In addition to evaluating drug resistance properties, single cell analysis is also important for characterization of the mechanical properties of cancer cells. The mechanical properties of cells are phenotypical markers of their biophysical state which can inform on cytoskeletal and nuclear organization^{1,2}, cell cycle stage^{3,4}, degree of differentiation^{5,6} and pathological outcomes^{7,8}. Therefore, mechanical characterization of cells is of paramount significance in understanding cellular function, characterizing disease states and potentially correlating disease progression to patient outcomes.

Traditional techniques such as micropipette aspiration³⁰ for characterizing mechanical properties are laborious and have low throughput. Alternatively, deformability cytometry techniques^{1,27-29} provide higher throughput but impose extremely fast deformation on cells that is not physiologically relevant. Besides, none of the available mechanical characterization techniques are able to phenotype cells on subcellular level due to the technical difficulties in combining mechanical and fluorescent measurements at single cell level.

In this study, we present a microfluidic device and method that enables us to capture thousands of single cells at defined locations in a microgroove or microtube (FIG. 1B). These microgrooves or microtubes are analogous to traditional glass microcapillary pipettes that capture individual cells. We therefore dub the device design as microfluidic pipette aspirators (MFA). The unique design features are a fluidic network where the inlet channels have a dead-end forcing fluid to go into the microgrooves or microtubes, ensuring that cells have a chance to get captured in their respective locations. In addition, the inlet channels have a large height than the microgrooves or microtubes to ensure uniform pressure distribution.

Based on the geometry of the microgroove or microtubes, individual target cells or clusters of cells can be captured. The microtube has a pillar obstacle that bifurcates the incoming flow allowing capture of cells. The gap between the pillar in the microtubes can be tuned to allow size-selective sorting and capture of target cells or clusters of cells from a mixed population of cells.

The MFA was tested for two applications—phenotyping drug response of individual cells, and mechanical characterization of cells. To determine the fate of individual cells exposed to drugs captured, they were captured in pillar-laden microtubes and tracked over time to see if they undergo apoptosis, or undergo mitosis and proliferate or simply do not divide over the time-scale of the experiment. The same method and device with microgrooves was also used to deform individual cells using fluid pressure enabling their mechanical characterization. Additionally, after the desired drug resistance and mechanical measurements, reagents can be introduced to fix cells, and stain specific molecular markers.

FIGS. 1A and 1B show a summary of the overall method and a detailed structure for use with the present invention. FIG. 1A is a Schematic of a standard drug assay and fate of the cancer cell after chemotherapy. FIG. 1B shows MCF-7 cells identified as apoptotic, quiescent and proliferating after 48-hour drug exposure.

The present microfluidic setup provides the following benefits: i) isolation of single cancer cells with no loss using a pipette, ii) tracking of the lineage of the isolated single cells without losing their primary identity, iii) identify the drug resisting cells and iv) classify them as apoptotic, proliferating and quiescent.

To test the technology, the cell lines MCF-7 and MB231 (breast cancer lines) and the FDA approved chemotherapeutic agent Doxorubicin (potent drug used in the treatment of breast cancer) was used. FIG. 1A shows a schematic of the proof of concept experimentation. FIG. 1A shows that initially in a comparative drug study, the fate of the live cells after exposure to different drug dosages would be monitored through the means shown in FIG. 1B. FIG. 1B shows the microfluidic device entrapping drug treated cells showing distinct cellular fates as apoptotic, proliferating and quiescent.

A solution of polydimethylsiloxane (PDMS) monomer and curing agent was mixed in the ratio of 10:1 and degassed. This degassed solution was poured on the master mold making a ˜6 mm thick layer. This master mold was placed for curing in an oven, set at 70° C. for two hours. After the PDMS was cured, it was cut using a scalpel and peeled off the mold. A 6 mm diameter inlet reservoir and a 1 mm diameter outlet reservoir were punched using a biopsy punch (Miltex, Japan).

The PDMS device was bonded to the glass slide by plasma bonding. Firstly, the PDMS replica and the cover glass (Thermo Scientific™ Richard-Allan Scientific™, 24×50 mm) were cleaned using isopropanol. These cleaned PDMS chip and cover glass were placed in the plasma cleaner (PDC-32G, Harrick Plasma) and the bonding surfaces were activated with the air plasma for 90 seconds. After bonding the PDMS chip to the cover glass, the microfluidic device was placed in the oven at 70° C. for 4 minutes for strengthening the bonding. After the device was made, a frustum was cut out of 1000 μL pipette tip (Fisher Scientific) at the graduation volumes 500 μL and 1000 μL and it was snug fit to the inlet of the microfluidic chip for maintain a constant hydrodynamic flow inside the microfluidic device. This microfluidic device is filled with phosphate buffer saline (PBS, Gibco) for maintaining the hydrophilic nature of the microfluidic channels.

Cell culture. The breast cancer cell lines MCF-7 (ATCC #HTB-22) and MDA-MB-231 (ATCC #HTB-26) were obtained from American Type Cell Collection (ATCC). MCF-7 and MB-231 cells were cultured in Dulbecco's Modified Eagle Medium (DMEM, Gibco) containing 10% Fetal Bovine Serum (FBS, ATCC), 1% Penicillin/Streptomycin (Gibco) and 1% sodium pyruvate (Gibco). The cells were incubated at 37° C. in a 5% CO₂ environment. The confluent cells were collected for experiment using Trypsin/EDTA (0.25%, Gibco). Multiple cell concentrations of MCF-7 were used for optimizing cell concentration. An initial concentration of 1×10⁶ cells/ml was diluted to a desired concentration for all experiments.

Cell Sample Preparation. To achieve maximum single cell trapping efficiency in the microfluidic device, the inventors used a cell concentration of 0.02×10⁶ cells/ml. The dilution and staining of the cells were done using pipette, followed by vortex mixing to avoid clusters of cells. These stained cells were further incubated at 37° C. for different time periods, depending on the incubation period of the dye.

Drug Assays. Drug assays were conducted on the tumor cells trapped in the microfluidic device. Food and Drug Administration (FDA) approved chemotherapeutic medication Doxorubicin hydrochloride (Dox, Sigma Aldrich) was used on these cells. Dox was diluted with WFI (water for injection) for cell culture (Gibco) to make a 1 mM Dox solution and it was stored at 4° C. This stock solution of Dox was diluted to 100 μM using DMEM, and further 10-fold serial dilutions were done using DMEM or 1× Annexin Binding Buffer (Life technologies) to obtain the desired concentrations of 100 μM, 10 μM, 1 μM, 0.1 μM, 0.01 μM and 0.001 μM. For all experiments, Dox was prepared freshly by diluting it to 100 μM using DMEM and further dilutions were done to achieve the desired concentrations of Dox. The mixing of Dox with the cell samples and loading them in the microfluidic device was done within 2 minutes, followed by imaging of the loaded sample in the microfluidic device. After imaging the microfluidic device, it was incubated in 37° C. and 5% CO₂ environment.

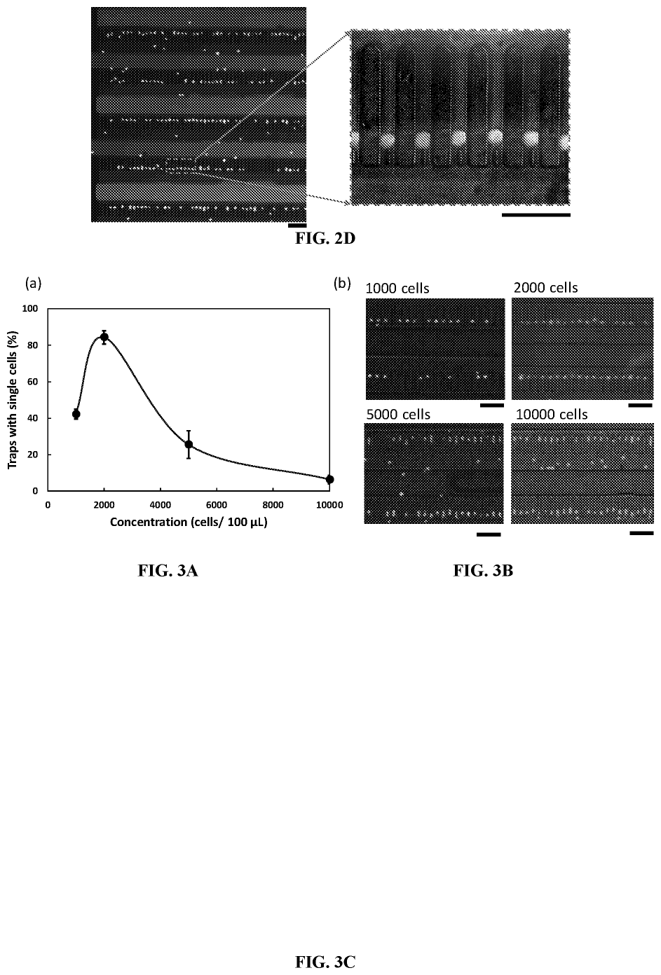

Experimental Protocol. The experimental setup for single cell isolation consists of the microfluidic device, 200 μL single channel pipette (Eppendorf) and a motorized inverted system microscope (Olympus IX81). A sample of 2000 cells in 100 μL was loaded in the 6 mm reservoir (inlet) in the microfluidic device. A 200 μL single channel pipette was set at the 1 mm outlet and it was used to aspirate the cell sample. Flow is generated from the inlet reservoir due to the aspiration pressure and passes through the trapping array. As soon as a cell enters into the parking spots, it gets trap there due to the obstacle pillar. This setup is shown the by schematic in FIG. 2A.

The MCF-7 cells are tagged using a green fluorescent dye CMFDA (Life technologies) and are trapped in the microfluidic device using the same protocol. FIG. 2D shows a zoomed out (4× objective) and 10-fold magnified image of trapped MCF-7 cells. Media was added in the hydrodynamic height added on the reservoir to keep the cells from getting displaced from the parking spots. All the cell samples including the drug treated cells were handled with the same experimental procedure. The loading time of the cells after cell sample preparation is ˜5 seconds. The hydrodynamic height for the drug treated samples was given by media of corresponding drug concentration.

Cell staining, viability and apoptosis detection. Proliferation of cells in the microfluidic chip was monitored using CellTracker™ Green CMFDA dye (Life technologies). These counts were validated without staining and by using NucBlue™ Live ReadyProbes™ Reagent (Life technologies) to identify the cell nuclei and thus quantifying the cell counts. LIVE/DEAD® Cell Imaging Kit (Life technologies) was used to analyze viability of cells. The stain was made according to the manufacturer's protocol and was used to identify the live and dead cells. Alexa Fluor® 488 Annexin V (Life technologies) was used for detection of apoptosis. The cell sample and Dox dilution was done using 1× Annexin Binding Buffer (Life technologies). 100 μL of sample was prepared (80 μL cell sample+10 μL Annexin V+10 μL Dox) for conducting drug assays. The final concentrations of the Dox in 100 μL sample were 0.01 μM, 0.001 μM and 0.0001 μM. This sample was incubated at 37° C. and 5% CO₂ for 30 minutes before loading in the microfluidic device. The images were taken at different time points for different studies. The microfluidic device was stored in the incubator at 37° C. and 5% CO₂ between different image time points.

Image acquisition and processing. All the imaging for this study was done using Olympus IX81 microscope (Massachusetts, USA) and Hamamatsu digital camera (ImagEM X2 EM-CCD, New Jersey, USA). The microscope was equipped with a Thorlabs automated stage (New Jersey, USA) and was controlled by the software Slidebook 6.1 (3i Intelligent Imaging Innovations Inc., Denver, USA). The trapped cells were imaged under brightfield d and TRITC, FITC, DAPI fluorescent filters with exposure times between 10 ms to 380 ms. Images were processed using ImageJ (https://imagej.nih.gov/ij/).

Influence of cell concentration on trapping. For trapping cells in the microfluidic device, the cells were aspirated from the outlet using a 200 μL single channel pipette. As the channel width of the trap was 16 μm, almost same as the size of MCF-7 and MB-231 cancer cells (15.77±1.1 μm); it was assumed that a cell will occupy the trap.⁴¹ The pillars avoid the cells from flowing out of the trap during cell loading. As there is no bypass channel in this microfluidic device, all the cells at the inlet get trapped inside the device after aspiration. Thus, different concentrations of cells were loaded in the inlet to optimize the single cell trapping. Concentrations such as 10000 cells/100 μL, 5000 cells/100 μL, 2000 cells/100 μL and 1000 cells/100 μL were used to analyze the trapping of single cells. The efficiency of single cell trapping is calculated as

$$Efficiency of single cell trapping = \frac{No . of single cells trapped}{Total no . of traps} \times 100$$

As shown in FIG. 3A, as the concentration of cells increases the efficiency of single cell trapping decreases. Also, at very low concentrations such as 1000 cells/100 μL, single cell trapping is low. FIG. 3B shows different concentrations of CMFDA tagged cells in the microfluidic device. It can be seen from these figures that at lower concentration of 1000 cells/100 μL, several traps remain empty, thus the device is not being used at its full capacity. Whereas at higher concentrations (10000 cells/100 μL and 5000 cells/100 μL), the traps are occupied with two or more cells, hence leading to loss of individuality of the trapped cells. FIG. 3C shows the distribution of cells in each trap at varying concentrations. This distribution concurs with empty and over occupied traps of FIG. 3B.

The results from FIGS. 3A and 3B leads the inventors to find the most optimum concentration for achieving maximum number of single cells. The selection of optimum concentration was based on two factors: maximum single cell isolation and minimum empty traps. Therefore, the concentration of 2000 cells/100 μL was found to be the most optimum for this microfluidic device with trapping efficiency >85%.

As CTCs are rare, ranging from 5-1281 CTCs per mL of blood depending on the stage of cancer,⁴² it is extremely important to avoid cell loss. Thus, this microfluidic technique for single cell isolation utilizes ˜98% of the loaded sample by using only a pipette as a tool for liquid handling.⁴³

Tracking the lineage of single cells. To illustrate the utility of the microfluidic device, in this section the inventors demonstrate lineage tracking of MCF-7 breast cancer cells and the effect of Dox on their proliferation potential.

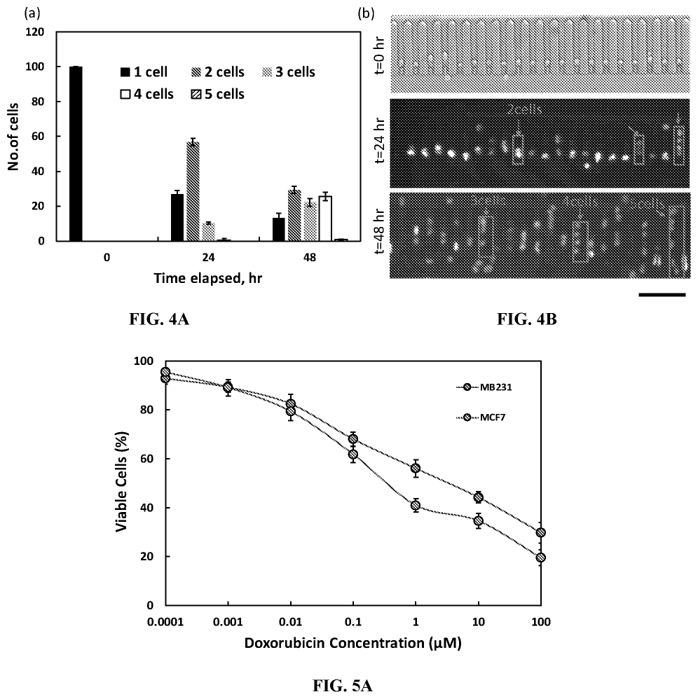

As the single cells are trapped in long and narrow channels with a continuous supply of fresh media, they are restrained to grow in a linear pattern inside the channels. The microfluidic chip is imaged at multiple time points to evaluate the growth of single cells. As shown in FIG. 4A at time t=0 hr, all the traps containing single cells are collectively considered to be 100%. After 24 hours, ˜50% of these single cells divide into two cells, ˜10% divide into 3 cells and a very small population of single cells divides into 4 cells. These cells further undergo division and the results for proliferation of single cells up until 48 hours is as shown in FIG. 4A. Almost 15% of the single cells do not undergo cell division in 48 hours. These 15% of cells can be classified as apoptotic or quiescent. FIG. 4B shows the cells imaged at three different time points with DAPI nuclear stain.

Thus, this microfluidic device can track the lineage of cells without losing its singularity.

Drug susceptibility of single cells. Evolution of a single cell can lead to the formation of heterogeneous lineages, which can result in the formation of a malignant tumor.^{19,44} This clonal diversity gives rise to intra-tumor heterogeneity; meaning variation in subpopulations within the tumor.^{45,46} Thus, evaluating the effect of drugs on these cells can give important information about the drug resistance in cancer cells.⁹

Given that the inventors established that single cells can be trapped, and the lineage of these trapped single cells can be tracked in the microfluidic device, the inventors studied the effect of Doxorubicin on individual breast cancer cells. Doxorubicin belongs to anthracycline family of anti-cancer drugs' and induces apoptosis in cancer cells via different mechanisms of action; such as regulated intramembrane proteolysis⁴⁸, inducing DNA damage by free radical formation⁴⁹, avoiding DNA crosslinking by breaking DNA single strands resulting in inhibition of macromolecular biosynthesis^{50,51}, hindering topoisomerase^{51,52}.

Drug resistance is a major challenge in cancer therapy. Although Doxorubicin has multiple mechanisms for inducing apoptosis in cancer cells, several cells show drug resistance and escape cell death.^{2,53} Single cell analysis can be maneuvered in addressing the heterogeneity in cancer cells by studying how different therapies affect various CTCs; thus, identifying drug resistant cells. Resistance in responding to chemotherapies can be classified as: (i) intrinsic resistance (ii) acquired resistance. Intrinsic resistance is inherently present prior to chemotherapy; so, the CTCs show no response to the primary treatment. Whereas, acquired resistance is developed only during or subsequently after the treatment.^{54,55} This information would be helpful in detecting resistance promptly, in predicting treatment efficacy and developing precision medicine for targeting CTCs.⁵⁶

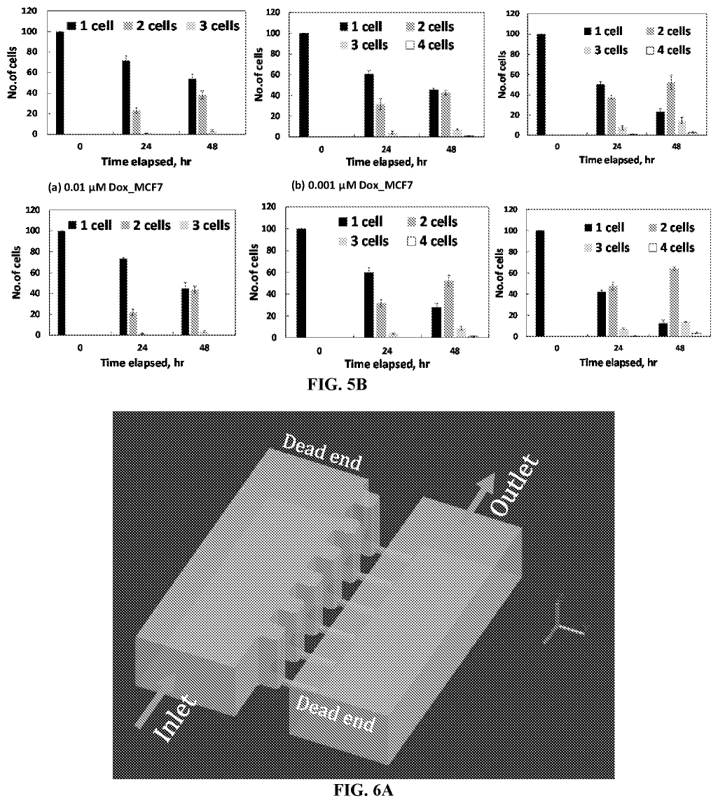

To understand the effect of different concentrations of dox on viability of single cells, the inventors conducted a dose response cell viability assay in the microfluidic device. FIG. 5A shows the dose response curve of MCF-7 and MB-231 breast cancer cells after 24 hours of administration of dox. From this viability curve, three concentrations were selected to study the proliferation potential of drug resistant MCF-7 and MB-231 single cells. As the lowest dosage of doxorubicin has the strongest impact on the proliferation rate, the lowest three concentrations were selected for this study. 0.01 μM, 0.001 μM and 0.0001 μM concentrations of Dox were selected for this study as the viability of single cells in these concentrations was maximum. FIG. 5B shows the proliferation potential of MCF-7 and MB-231 single cells in the presence of Dox over a period of 48 hours. With decrease in concentration of Dox, the number of proliferating cells is increasing. For MB-231 and 0.01 μM dox, ˜20% of cells proliferated in 24 hours and ˜40% cells proliferated at the end of 48 hours. Similarly, for MB-231 and 0.001 μM dox, ˜50% cells proliferated; and for MB-231 and 0.0001 μM dox ˜70% cells proliferated. Furthermore, for MCF-7 and 0.01 μM, 0.001 μM and 0.0001 μM concentrations of Dox, ˜45%, ˜60% and ˜80% cells underwent proliferation. Thus, cancer cells show drug resistance by proliferating in the presence of drug and as this microfluidic device is capable of identifying these proliferative drug resistant cells, it can aid in developing a targeted therapy for these drug resistant cells.

Identifying the state of drug treated cells. From FIGS. 5A and 5B, the proliferated cells give some information about the drug resistance of the cells, but it does not give complete information about the state of single cells. These single cells can exist in an apoptotic state or a non-proliferative (quiescent) state. Thus, identifying the state of the cell can give overall information about the drug resistance of cancer cells.

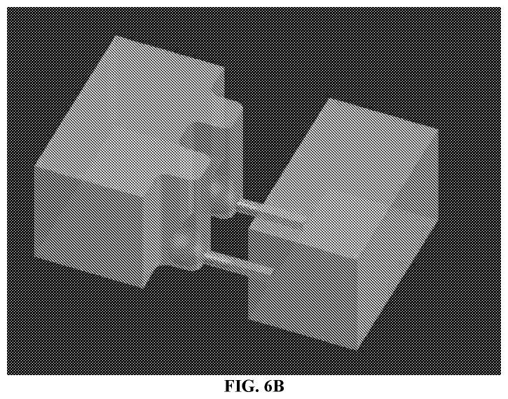

As shown in FIGS. 6A and 6B, the drug treated MCF-7 and MB-231 cells are categorized as apoptotic, non-proliferating and proliferating for three different concentrations of Dox. The apoptotic cells are dead cells; and the non-proliferating cells are the drug resistant cells which are live and have very low proliferative potential. The proliferating cells are also drug resistant cells, which multiply despite of the presence of drug. The drug response for these trapped cells is assessed at 0, 6, 12, 24, 48 hours at 0.01 μM, 0.001 μM and 0.0001 μM concentrations of Dox.

As the concentration of the drug decreases, there is an increase in the number of proliferative cells and decrease in number of the apoptotic cells. However, no definitive trend is seen in the non-proliferative cells. For MCF-7 and 0.01 μM dox, 0.001 μM and 0.0001 μM; ˜60%, ˜75%, ˜90% cells show drug resistance respectively. Similarly, for MB-231 and 0.01 μM dox, 0.001 μM and 0.0001 μM; ˜60%, ˜80%, ˜85% cells show drug resistance respectively.

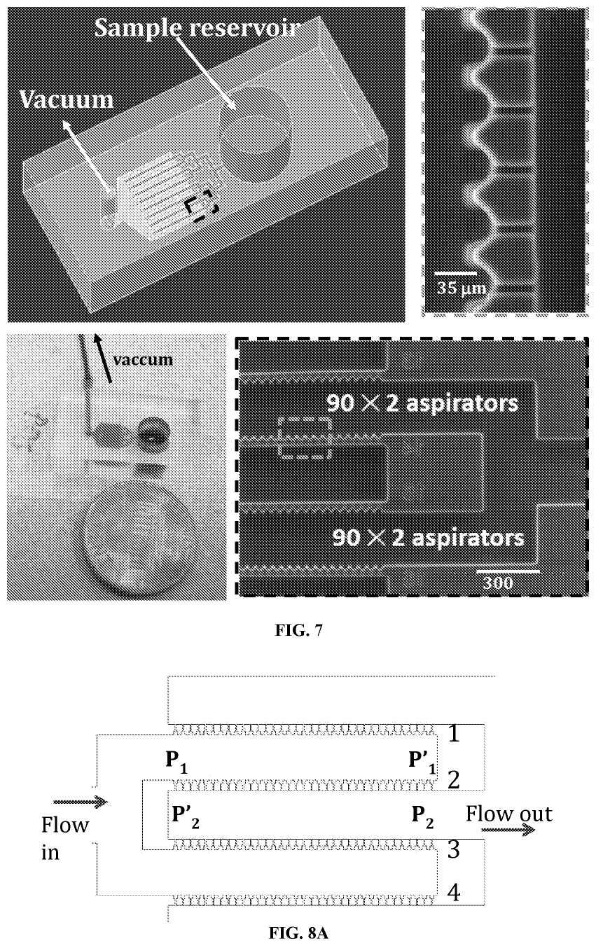

At the end of 48 hours, the non-proliferative cells were identified as quiescent cells. Quiescent cells are the cells showing a reversible non-proliferative fate³⁰ which can prove to be more lethal in the relapse or progression of cancer.⁵⁷ FIG. 7 shows the three categorical fates of the cancer cells namely apoptotic, quiescent and proliferative at 48 hours using three different concentrations of dox 0.01 μM, 0.001 μM and 0.0001 μM. From this data, 8%, 10% and 6% of cells were identified as quiescent for 0.01 μM, 0.001 μM and 0.0001 μM of dox and MCF-7. Similarly, 23%, 34% and 10% of cells were identified as quiescent for 0.01 μM, 0.001 μM and 0.0001 μM of dox and MB-231.

As this microfluidic device tracks the lineage of every individual cell without losing its singularity over a period, a precise chemotherapy can be designed for these drug resistant cells resulting in achieving higher treatment efficiency.

The present invention includes the microfluidic device and its use for single cell isolation and phenotypic detection. Briefly, a microfluidic set-up is described, cell concentration optimization determined, single cell proficiency and proliferation is measured, and lastly drug susceptibility is tested in cells to look at proliferation and/or changes to the cellular cytoskeleton are measured.

The microfluidic device has a two-layer design that was fabricated using photoresist (SU-8 2015 and SU-8 2050, Microchem Corporation, 2100 rpm and 1650 rpm spin-speed, 20 μm and 100 μm thick respectively). FIG. 2A above, demonstrates the schematic layout of the microfluidic device. In the device the first layer is comprised by trapping arrays, the second layer has the inlet and outlet channels. Within the first layer, there are 16 columnar trapping arrays, each with 100 trapping spots. Overall, there are 1600 traps in the device. Since the end of each columnar array is closed, all liquid and cells need to pass through the trapping array. Moreover, the ends of the columnar arrays are closed; all liquid and cells need to pass through the trapping array. The inlet and outlet channels can be appreciated through scanning electron microscopy (SEM) on FIG. 2C, FIG. 2B shows the microfluidic device filled with red food dye along with a penny for comparison.

A component of the operating protocol is the cell sample preparation, to achieve the maximum single cell trapping efficiency in the microfluidic device; a concentration of 0.02×10⁶ cells/ml was used. For the drug assay, the assays were conducted on the tumor cells that were trapped in the microfluidic device. Doxorubicin (FDA approved) was used on the cells.

To understand the effect of different concentrations of dox on the viability of single cells, the inventors conducted a dose response cell viability assay in the present invention. FIG. 5A shows the dose response cell viability assay in our microfluidic device of both MCF-7 and MB-231 breast cancer cell lines after 24 hours of administration. From this viability curve, three concentrations were selected to study the proliferation potential of drug resistant MCF-7 and MB-231 single cells. Dosages of 0.01 μL, 0.001 μL and 0.0001 μL concentrations of Dox were selected for this study since the viability of the cells at these concentrations was maximum.

FIG. 7 shows different views of another design of the device of the present invention that enables characterization of mechanical properties of individual cells and correlating mechanical properties with molecular marker expression. FIG. 7 includes an isometric view that includes the source of liquid, the array of cells, and the vacuum or outlet for the liquid, shows a close-up view of the microgroove/microtube for isolating single cells that include chambers that help drive cells into the microgrooves/microtubes, shows the relative size of the device when compared to a penny, and a broader view showing the arrays and aspirators for drawing the cells into the microgrooves/microtubes. Total 1440 traps, 150 μL Inlet reservoir, Aspiration channel dimension 5×5×35 μm³. In these figures, a schematic of the complete design. It has an inlet reservoir for holding cell sample. The inlet channel divides into 8 channels and all of them have dead ends. Aspiration traps are place on both sides of the inlet channels. Outlets of two aspiration arrays comes into an outlet channel. Except the first and last outlet channel where a single aspiration has outlets. It has total 1440 traps. This is the snapshot of the PDMS device. In the SEM image you can have a sense of the larger dimension of inlet and outlet channels compared to the aspiration channels. To ensure that pressures are actually nearly same for all the traps, CFD simulations were conducted, as summarized in the following figures.

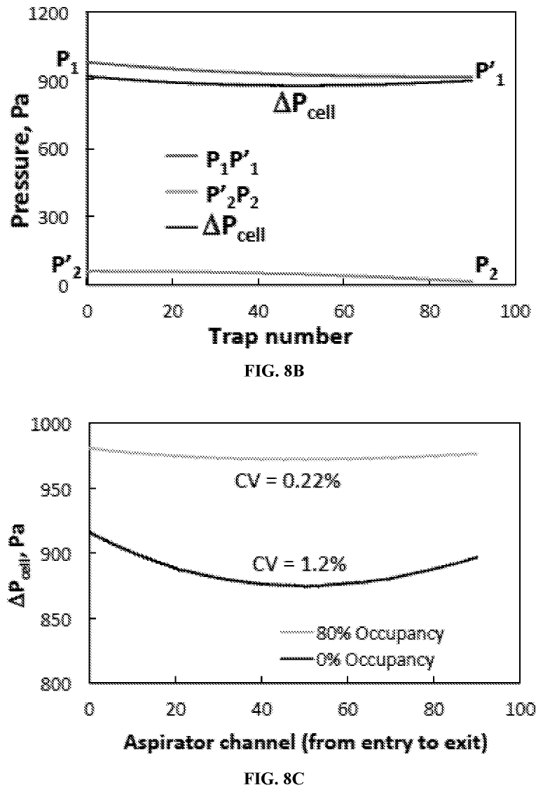

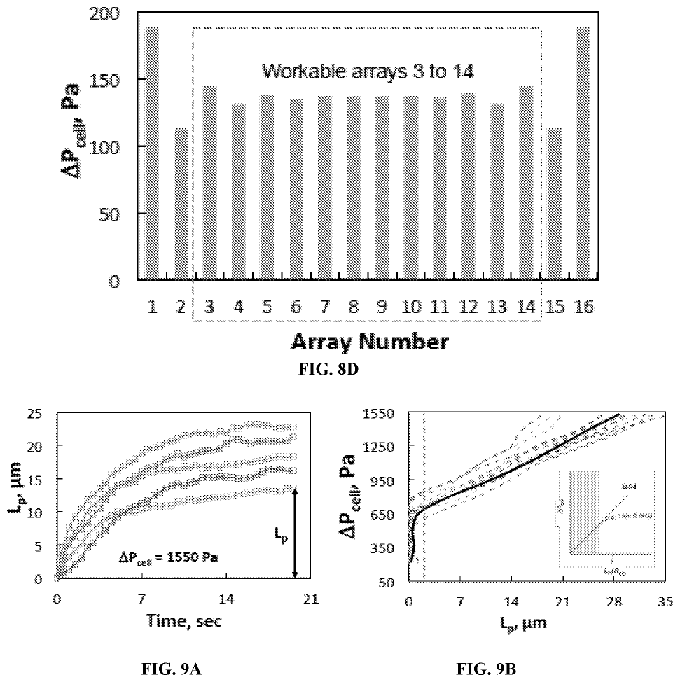

FIGS. 8A to 8D are a side view with the inflow and outflow of the device. FIG. 8A shows the locations for pressure and other measurements from simulations of the device. FIG. 8B is a graph that shows the pressure at the various trap numbers. FIG. 8C is a graph that shows the change in pressure versus channel of aspiration. FIG. 8D is a graph that shows the change in pressure and the array number. The pressure distribution versus trap number for a representative array number 2 is shown when there is no cell present. P₁P_{1′} is the inlet channel pressure distribution and p_{2′}P₂ is the outlet channel pressure distribution. And delta p cell is the difference between inlet and outlet pressures. It was found that the maximum coefficient of variation in ΔP_{cell} was only 1.2%. This CV decreases as the cells are trapped. If all the traps are filled with cells there will be no flow in the device and ΔP_{cell} will be exactly same for all the cells. The inventors also determined whether all the arrays have same pressure drop as first and last outlet channel get outlets from one aspiration array. It was found that aspiration array 1, 2, 15 and 16 have different pressure drops. Workable arrays were 3 to 14. The following figures take a look at the cell loading process. The breast cancer cell line MDA-MB231 was selected to characterize the MFA device.

FIGS. 9A and 9B are graphs that show rheological cell-model selection simulations. FIG. 9A is a graph that shows the length versus time. FIG. 9B is a graph that shows the change in pressure versus length. Thus, it was found that the deformation is in linear regime, MDA-MB231 behaves like a solid object, and the working driving pressure 650 Pa to 1550 Pa.

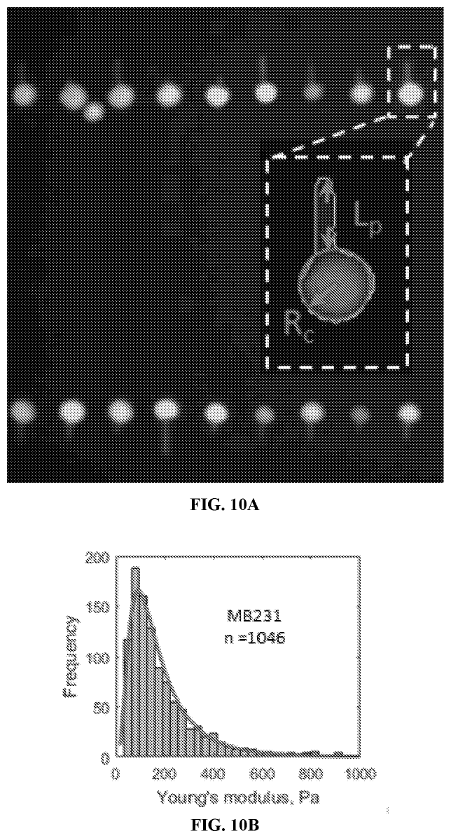

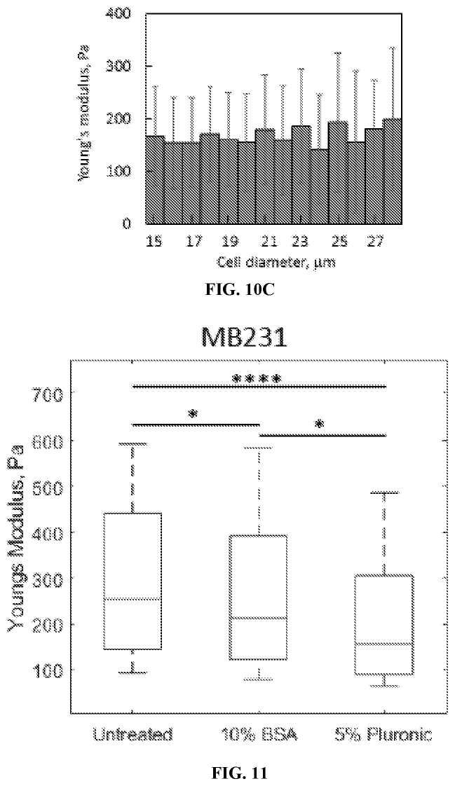

FIGS. 10A to 10C show the mechanical characterization of cells in the device of the present invention. FIG. 10A shows a top-view of cells in the microgrooves/microtubes of the present invention. FIG. 10B is a graph that shows the frequency of cells versus the Young's modulus of the cells. FIG. 10C is a graph that shows the Young's modulus of the cells versus the cell diameters. These graphs show that Breast cancer cell line MDA-MB231, ΔP=700 Pa, and shows that the measured Young's modulus is independent of cell size.

FIG. 11 is a graph that shows the Young's modulus of MB231 cells, when cells are untreated, in 10% BSA, and 5% pluronic. It was found that 5% Pluronic treated device measures 40% less Young's modulus than untreated, thus, Friction has statistically significant contribution on measurements.

FIGS. 12A and 12B show the sensitivity of different cells (MB231 and MCF7) to different drugs (blebbistatin and paclitaxel), and the Young's modulus of the cells. FIG. 12A is a graph that shows the Young's modulus of MB231 cells under control conditions, treated with blebbistatin or paclitaxel. FIG. 12B is a graph that shows the Young's modulus of MCF7 cells showing whether they are in G1, S, or G2 phase. Thus, the cytoskeletal alternations in MB231 are induced by drug blebbistatin and paclitaxel. Also, cell cycle phases have altered cytoskeletal scaffold. Thus, the μFPA device is sensitive enough to measure cytoskeletal alterations caused by drug interventions and cell cycle phase.

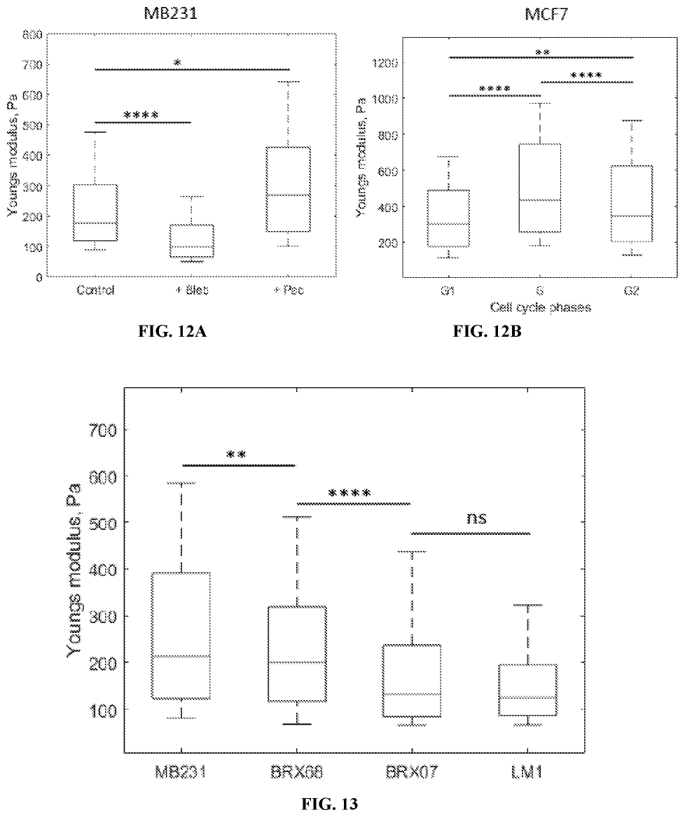

FIG. 13 is a graph that shows the Young's modulus of CTC-derived cell lines: MB231, BRX68, BRX07, and LM1. Based on these results, CTC-derived cell lines are softer than the in vitro cell line MB231, which can be measured and calibrated using the present invention.

In conclusion, the present inventors developed and characterized a high throughput μFPA. Using the present invention it was possible to determine the contribution of friction on measurements and cell-to-cell variability. It was also shown that using the present invention it was possible to test the sensitivity of μFPA to very small cytoskeletal perturbations. Finally, it was also possible to measure the different stiffness of CTC derived cell lines.

It is contemplated that any embodiment discussed in this specification can be implemented with respect to any method, kit, reagent, or composition of the invention, and vice versa. Furthermore, compositions of the invention can be used to achieve methods of the invention.

It will be understood that particular embodiments described herein are shown by way of illustration and not as limitations of the invention. The principal features of this invention can be employed in various embodiments without departing from the scope of the invention. Those skilled in the art will recognize, or be able to ascertain using no more than routine experimentation, numerous equivalents to the specific procedures described herein. Such equivalents are considered to be within the scope of this invention and are covered by the claims.

All publications and patent applications mentioned in the specification are indicative of the level of skill of those skilled in the art to which this invention pertains. All publications and patent applications are herein incorporated by reference to the same extent as if each individual publication or patent application was specifically and individually indicated to be incorporated by reference.

The use of the word “a” or “an” when used in conjunction with the term “comprising” in the claims and/or the specification may mean “one,” but it is also consistent with the meaning of “one or more,” “at least one,” and “one or more than one.” The use of the term “or” in the claims is used to mean “and/or” unless explicitly indicated to refer to alternatives only or the alternatives are mutually exclusive, although the disclosure supports a definition that refers to only alternatives and “and/or.” Throughout this application, the term “about” is used to indicate that a value includes the inherent variation of error for the device, the method being employed to determine the value, or the variation that exists among the study subjects.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps. In embodiments of any of the compositions and methods provided herein, “comprising” may be replaced with “consisting essentially of” or “consisting of”. As used herein, the phrase “consisting essentially of” requires the specified integer(s) or steps as well as those that do not materially affect the character or function of the claimed invention. As used herein, the term “consisting” is used to indicate the presence of the recited integer (e.g., a feature, an element, a characteristic, a property, a method/process step or a limitation) or group of integers (e.g., feature(s), element(s), characteristic(s), property(ies), method/process steps or limitation(s)) only.

The term “or combinations thereof” as used herein refers to all permutations and combinations of the listed items preceding the term. For example, “A, B, C, or combinations thereof” is intended to include at least one of: A, B, C, AB, AC, BC, or ABC, and if order is important in a particular context, also BA, CA, CB, CBA, BCA, ACB, BAC, or CAB. Continuing with this example, expressly included are combinations that contain repeats of one or more item or term, such as BB, AAA, AB, BBC, AAABCCCC, CBBAAA, CABABB, and so forth. The skilled artisan will understand that typically there is no limit on the number of items or terms in any combination, unless otherwise apparent from the context.

As used herein, words of approximation such as, without limitation, “about”, “substantial” or “substantially” refers to a condition that when so modified is understood to not necessarily be absolute or perfect but would be considered close enough to those of ordinary skill in the art to warrant designating the condition as being present. The extent to which the description may vary will depend on how great a change can be instituted and still have one of ordinary skill in the art recognize the modified feature as still having the required characteristics and capabilities of the unmodified feature. In general, but subject to the preceding discussion, a numerical value herein that is modified by a word of approximation such as “about” may vary from the stated value by at least ±1, 2, 3, 4, 5, 6, 7, 10, 12 or 15%.

All of the compositions and/or methods disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods and in the steps or in the sequence of steps of the method described herein without departing from the concept, spirit and scope of the invention. All such similar substitutes and modifications apparent to those skilled in the art are deemed to be within the spirit, scope and concept of the invention as defined by the appended claims.

To aid the Patent Office, and any readers of any patent issued on this application in interpreting the claims appended hereto, applicants wish to note that they do not intend any of the appended claims to invoke paragraph 6 of 35 U.S.C. § 112, U.S.C. § 112 paragraph (f), or equivalent, as it exists on the date of filing hereof unless the words “means for” or “step for” are explicitly used in the particular claim.

For each of the claims, each dependent claim can depend both from the independent claim and from each of the prior dependent claims for each and every claim so long as the prior claim provides a proper antecedent basis for a claim term or element.

### REFERENCES

1 Giaccone, G. & Pinedo, H. M. Drug Resistance. The oncologist 1, 82-87 (1996).
2 Links, M. & Brown, R. Clinical relevance of the molecular mechanisms of resistance to anti-cancer drugs. Expert reviews in molecular medicine 1999, 1-21, doi:10.1017/s1462399499001099x (1999).
3 Volm, M. & Efferth, T. Prediction of Cancer Drug Resistance and Implications for Personalized Medicine. Frontiers in oncology 5, 282-282, doi:10.3389/fonc.2015.00282 (2015).
4 Holohan, C., Van Schaeybroeck, S., Longley, D. B. & Johnston, P. G. Cancer drug resistance: an evolving paradigm. Nature Reviews Cancer 13, 714, doi:10.1038/nrc3599 (2013).
5 Mansoori, B., Mohammadi, A., Davudian, S., Shirjang, S. & Baradaran, B. The Different Mechanisms of Cancer Drug Resistance: A Brief Review. Advanced pharmaceutical bulletin 7, 339-348, doi:10.15171/apb.2017.041 (2017).
6 Housman, G. et al. Drug resistance in cancer: an overview. Cancers 6, 1769-1792, doi:10.3390/cancers6031769 (2014).
7 Leary, M., Heerboth, S., Lapinska, K. & Sarkar, S. Sensitization of Drug Resistant Cancer Cells: A Matter of Combination Therapy. Cancers 10, 483, doi:10.3390/cancers10120483 (2018).
8 Sachs, J. R., Mayawala, K., Gadamsetty, S., Kang, S. P. & de Alwis, D. P. Optimal Dosing for Targeted Therapies in Oncology: Drug Development Cases Leading by Example. Clinical Cancer Research 22, 1318, doi:10.1158/1078-0432.CCR-15-1295 (2016).
9 Gatenby, R. A. A change of strategy in the war on cancer. Nature 459, 508, doi:10.1038/459508a (2009).
10 Lippert, T. H., Ruoff, H.-J. & Volm, M. Intrinsic and Acquired Drug Resistance in Malignant Tumors. Arzneimittelforschung 58, 261-264, doi:10.1055/s-0031-1296504 (2008).
11 Terzi, M. Y., Izmirli, M. & Gogebakan, B. The cell fate: senescence or quiescence. Molecular biology reports 43, 1213-1220, doi:10.1007/s11033-016-4065-0 (2016).
12 Rodier, F. & Campisi, J. Four faces of cellular senescence. The Journal of Cell Biology 192, 547, doi:10.1083/jcb.201009094 (2011).
13 Nakamura-Ishizu, A., Takizawa, H. & Suda, T. The analysis, roles and regulation of quiescence in hematopoietic stem cells. Development 141, 4656, doi:10.1242/dev.106575 (2014).
14 Yao, G. Modelling mammalian cellular quiescence. Interface focus 4, 20130074, doi:10.1098/rsfs.2013.0074 (2014).
15 Kuilman, T., Michaloglou, C., Mooi, W. J. & Peeper, D. S. The essence of senescence. Genes & development 24, 2463-2479, doi:10.1101/gad.1971610 (2010).
16 Tellez-Gabriel, M., Ory, B., Lamoureux, F., Heymann, M.-F. & Heymann, D. Tumour Heterogeneity: The Key Advantages of Single-Cell Analysis. International Journal of Molecular Sciences 17, 2142, doi:10.3390/ijms17122142 (2016).
17 Fidler, I. J. & Hart, I. R. Biological diversity in metastatic neoplasms: origins and implications. Science 217, 998 (1982).
18 Fidler, I. J. Tumor heterogeneity and the biology of cancer invasion and metastasis. Cancer Res 38, 2651-2660 (1978).
19 Salmon, S. E. Human tumor colony assay and chemosensitivity testing. Cancer treatment reports 68, 117-125 (1984).
20 Lindhagen, E., Nygren, P. & Larsson, R. The fluorometric microculture cytotoxicity assay. Nat Protoc 3, 1364-1369, doi:10.1038/nprot.2008.114 (2008).
21 Takamura, Y. et al. Prediction of chemotherapeutic response by collagen gel droplet embedded culture-drug sensitivity test in human breast cancers. International Journal of Cancer 98, 450-455, doi:10.1002/ijc.10208 (2002).
22 Weisenthal, L. M. & Kern, D. H. Prediction of drug resistance in cancer chemotherapy: the Kern and DiSC assays. Oncology (Williston Park, N.Y.) 5, 93-103; disc. 104, 111-104, 117-108 (1991).
23 Galderisi, F. et al. Flow cytometric chemosensitivity assay as a predictive tool of early clinical response in acute lymphoblastic leukemia. Pediatric blood & cancer 53, 543-550, doi:10.1002/pbc.22119 (2009).
24 Petty, R. D., Sutherland, L. A., Hunter, E. M. & Cree, I. A. Comparison of MTT and ATP-based assays for the measurement of viable cell number. Journal of bioluminescence and chemiluminescence 10, 29-34, doi:10.1002/bio.1170100105 (1995).
25 Blumenthal, R. D. & Goldenberg, D. M. Methods and goals for the use of in vitro and in vivo chemosensitivity testing. Molecular Biotechnology 35, 185-197, doi:10.1007/BF02686104 (2007).
26 Grunberger, A., Wiechert, W. & Kohlheyer, D. Single-cell microfluidics: opportunity for bioprocess development. Current Opinion in Biotechnology 29, 15-23, doi:https://doi.org/10.1016/j.copbio.2014.02.008 (2014).
27 Young, E. W. K. & Beebe, D. J. Fundamentals of microfluidic cell culture in controlled microenvironments. Chemical Society reviews 39, 1036-1048, doi:10.1039/b909900j (2010).
28 Halldorsson, S., Lucumi, E., Gomez-Sjöberg, R. & Fleming, R. M. T. Advantages and challenges of microfluidic cell culture in polydimethylsiloxane devices. Biosensors and Bioelectronics 63, 218-231, doi:https://doi.org/10.1016/j.bios.2014.07.029 (2015).
29 Bithi, S. Engineering a drop-based microfluidic platform with the functionalities of well plates and pipetting systems Doctor of Philosophy thesis, Texas Tech University, (2013).
30 Kukhtevich, I. V., Belousov, K. I., Bukatin, A. S., Dubina, M. V. & Evstrapov, A. A. A microfluidic chip with hydrodynamic traps for in vitro microscopic investigations of single cells. Technical Physics Letters 41, 255-258, doi:10.1134/51063785015030086 (2015).
31 Carlo, D. D., Wu, L. Y. & Lee, L. P. Dynamic single cell culture array. Lab on a Chip 6, 1445-1449, doi:10.1039/B605937F (2006).
32 Kimmerling, R. J. et al. A microfluidic platform enabling single-cell RNA-seq of multigenerational lineages. Nature Communications 7, 10220, doi:10.1038/ncomms10220
https://www.nature.com/articles/ncomms10220#supplementary-information (2016).
33 Wlodkowic, D., Faley, S., Zagnoni, M., Wikswo, J. P. & Cooper, J. M. Microfluidic Single-Cell Array Cytometry for the Analysis of Tumor Apoptosis. Analytical Chemistry 81, 5517-5523, doi:10.1021/ac9008463 (2009).
34 Ahmmed, S. M. et al. Multi-sample deformability cytometry of cancer cells. APL Bioengineering 2, 032002, doi:10.1063/1.5020992 (2018).
35 Pang, L. et al. Construction of single-cell arrays and assay of cell drug resistance in an integrated microfluidic platform. Lab on a Chip 16, 4612-4620, doi:10.1039/C6LC01000H (2016).
36 Smith, L. et al. The analysis of doxorubicin resistance in human breast cancer cells using antibody microarrays. Molecular Cancer Therapeutics 5, 2115, doi:10.1158/1535-7163.MCT-06-0190 (2006).
37 Lao, J. et al. Liposomal Doxorubicin in the Treatment of Breast Cancer Patients: A Review. Journal of Drug Delivery 2013, 12, doi:10.1155/2013/456409 (2013).
38 Wang, S. et al. Doxorubicin induces apoptosis in normal and tumor cells via distinctly different mechanisms. intermediacy of H(2)O(2)- and p53-dependent pathways. The Journal of biological chemistry 279, 25535-25543, doi:10.1074/jbc.M400944200 (2004).
39 Lefley, D. V. et al. Antitumor Effects of Doxorubicin Followed by Zoledronic Acid in a Mouse Model of Breast Cancer. JNCI: Journal of the National Cancer Institute 100, 1167-1178, doi:10.1093/jnci/djn240 (2008).
40 Ahmmed, S. M. et al. Multi-sample deformability cytometry of cancer cells. APL Bioengineering 2, 032002, doi:10.1063/1.5020992 (2018).
41 Kamiyabi, N. Microfluidic Investigation of Biomechanics of Tumor Cells Under Microcirculatory Conditions PhD thesis, Texas Tech University, (2017).
42 Nagrath, S. et al. Isolation of rare circulating tumour cells in cancer patients by microchip technology. Nature 450, 1235-1239, doi:10.1038/nature06385 (2007).
43 Kong, F., Yuan, L., Zheng, Y. F. & Chen, W. Automatic Liquid Handling for Life Science: A Critical Review of the Current State of the Art. Journal of laboratory automation 17, 169-185, doi:10.1177/2211068211435302 (2012).
44 Shah, S. P. et al. The clonal and mutational evolution spectrum of primary triple-negative breast cancers. Nature 486, 395-399, doi:10.1038/nature10933 (2012).
45 Melo, F. D. S. E., Vermeulen, L., Fessler, E. & Medema, J. P. Cancer heterogeneity—a multifaceted view. EMBO reports 14, 686 (2013).
46 Fidler, I. J. & Hart, I. R. Biological diversity in metastatic neoplasms: origins and implications. Science 217, 998 (1982).
47 Aubel-Sadron, G. & Londos-Gagliardi, D. Daunorubicin and doxorubicin, anthracycline antibiotics, a physicochemical and biological review. Biochimie 66, 333-352 (1984).
48 Lal, M. & Caplan, M. Regulated intramembrane proteolysis: signaling pathways and biological functions. Physiology (Bethesda, Md.) 26, 34-44, doi:10.1152/physiol.00028.2010 (2011).
49 Bates, D. A. & Winterbourn, C. C. Deoxyribose breakdown by the adriamycin semiquinone and H2O2: evidence for hydroxyl radical participation. FEBS Lett 145, 137-142, doi:https://doi.org/10.1016/0014-5793(82)81222-2 (1982).
50 Wassermann, K. et al. Effects of 3′-deamino-3′-(3-cyano-4-morpholinyl)doxorubicin and doxorubicin on the survival, DNA integrity, and nucleolar morphology of human leukemia cells in vitro. Cancer Res 46, 4041-4046 (1986).
51 Gewirtz, D. A critical evaluation of the mechanisms of action proposed for the antitumor effects of the anthracycline antibiotics adriamycin and daunorubicin. Biochemical Pharmacology 57, 727-741, doi:https://doi.org/10.1016/S0006-2952(98)00307-4 (1999).
52 Tewey, K. M., Rowe, T. C., Yang, L., Halligan, B. D. & Liu, L. F. Adriamycin-induced DNA damage mediated by mammalian DNA topoisomerase II. Science 226, 466-468 (1984).
53 Giaccone, G. & Pinedo, H. M. Drug Resistance. The oncologist 1, 82-87 (1996).
54 Liang, S.-B. & Fu, L.-W. Application of single-cell technology in cancer research. Biotechnology Advances 35, 443-449, doi:https://doi.org/10.1016/j.biotechadv.2017.04.001 (2017).
55 Schmidt, F. & Efferth, T. Tumor Heterogeneity, Single-Cell Sequencing, and Drug Resistance. Pharmaceuticals 9, 33, doi:10.3390/ph9020033 (2016).
56 Zhu, W. et al. Next-generation molecular diagnosis: single-cell sequencing from bench to bedside. Cellular and Molecular Life Sciences 74, 869-880, doi:10.1007/s00018-016-2368-x (2017).
57 Vanner, R. J. et al. Quiescent sox2(+) cells drive hierarchical growth and relapse in sonic hedgehog subgroup medulloblastoma. Cancer cell 26, 33-47, doi:10.1016/j.ccr.2014.05.005 (2014).

1. A microfluidic device for single or multicell capture comprising:
a substrate;
one or more microgrooves or microtubes disposed within the substrate, each microgroove or microtube having a first end and a second end, wherein a width of the microgroove or microtube is a diameter of a target cell or a group of cells, wherein the microgroove or microtube comprises one or more chambers, wherein the microgroove or microtube has a constricted end and the microtube or microtube has a micropillar that bifurcates a fluid flow to capture a target cell or a groups of cells;
a fluid input disposed within the substrate in fluid communication with the first end of the one or more microgrooves or microtube; and
a fluid output disposed within the substrate in fluid communication with the second end of the one or more microgrooves or microtube, or the one or more chambers, wherein one or more cells that are captured in the microgroove can be analyzed as a single cell.

2. The microfluidic device of claim 1, wherein at least one of: the diameter of the target cell is between 1 to 100 μm in diameter; the one or more chambers are rectangular, circular, or triangular shaped; or a micropillar in the microtube is circular, rectangular or triangular shaped.

3. (canceled)

4. (canceled)

5. The microfluidic device of claim 1, wherein at least one of: the target cell is a cancer cell; or the target cell is a mammalian, plant, insect, or bacterial cell.

6. The microfluidic device of claim 1, further comprising an imaging device, wherein the imaging device detects one or more cells in the microgroove.

7. The microfluidic device of claim 1, wherein at least one of: the substrate is biocompatible or is a material coated to be biocompatible; or the substrate is selected from at least one of glass, silicon, polymer, plastic, metal, ceramic, semiconductor, or any combination thereof.

8. (canceled)

9. (canceled)

10. (canceled)

11. The microfluidic device of claim 1, wherein the microgrooves or microtubes further comprise at least one of: a portion that narrows to a size that is less than a diameter of a target cell to trap the target cell; are on a first plane, and at least one of the input or output are on a second plane; are sized to hold one or more target cells; are between about 1 to 75 μm and a height depth of the microgroove is between about 1 to 100 μm; can hold one or more daughter cells from target cells; or are on a first plane, and at least one of the input or output are on a second plane, wherein the input, the output, or both are above or below the first plane.

12. (canceled)

13. (canceled)

14. The microfluidic device of claim 1, wherein at least one of: a fluid drives the target cells or group of cells to enter the microgrooves or microtubes toward the fluid output or a fluid pressure drop is equally distributed among the microgrooves or microtubes.

15. (canceled)

16. (canceled)

17. The microfluidic device of claim 1, wherein a pillar gap in the microtubes is adjusted to capture cells of a larger size and let through smaller cells, to enable size-selective sorting of a mixed cellular population.

18. (canceled)

19. The microfluidic device of claim 1, wherein at least one of: the input comprises a first buffer exchange/feeding port or fluid reservoir; or the output port comprises a reservoir or a site for aspiration of a fluid in the device.

20. (canceled)

21. A method of making a microfluidic device having one or more microgrooves comprising:
providing a substrate;
forming one or more microgrooves or microtubes in or on the substrate, wherein each microgroove or microtube has a first end and a second end, wherein a width of the microgroove is a diameter of a target cell;
connecting a fluid input to the first end of the one or more microgrooves or microtubes; and
connecting a fluid output to the second end of the one or more microgrooves or microtubes, wherein one or more cells that are captured in the microgroove or microtubes can be analyzed as a single cell.

22. The method of claim 21, wherein at least one of: the diameter of the target cell is between 1 to 100 μm in diameter; the one or more chambers are rectangular, circular, or triangular shaped; or a micropillar in the microtube is circular, rectangular or triangular shaped.

23. The method of claim 21, wherein at least one of: the target cell is a cancer cell; or the the target cell is a mammalian, plant, insect, or bacterial cell.

24. The method of claim 21, further comprising an imaging device, wherein the imaging device detects one or more cells in the microgroove.

25. The method of claim 21, wherein at least one of: the substrate is biocompatible or is a material coated to be biocompatible; or the substrate is selected from at least one of glass, silicon, polymer, plastic, metal, ceramic, semiconductor, or any combination thereof.

26. (canceled)

27. (canceled)

28. (canceled)

29. The method of claim 21, wherein the microgrooves or microtubes further comprise at least one of: a portion that narrows to a size that is less than a diameter of a target cell to trap the target cell; are on a first plane, and at least one of the input or output are on a second plane; are sized to hold one or more target cells; are between about 1 to 75 μm and a height depth of the microgroove is between about 1 to 100 μm; can hold one or more daughter cells from target cells; or are on a first plane, and at least one of the input or output are on a second plane, wherein the input, the output, or both are above or below the first plane.

30. (canceled)

31. (canceled)

32. The method of claim 21, wherein at least one of: the input comprises a first buffer exchange/feeding port or fluid reservoir; or the output port comprises a reservoir or a site for aspiration of a fluid in the device.

33. (canceled)

34. A method of measuring cellular mechanical strength using a microfluidic device having one or more microgrooves comprising:
providing a microfluidic device for single or multicell capture comprising:
a substrate;
one or more microgrooves or microtubes disposed within the substrate, each microgroove or microtube having a first end and a second end, wherein a width of the microgroove or microtube is a diameter of a target cell or a group of cells, wherein the microgroove or microtube comprises one or more chambers;
a fluid input disposed within the substrate in fluid communication with the first end of the one or more microgrooves or microtubes; and
a fluid output disposed within the substrate in fluid communication with the second end of the one or more microgrooves or microtubes, or the one or more chambers, wherein one or more cells that are captured in the microgroove can be analyzed as a single cell; and
directing an imaging device to the one or more cells, such that the imaging device detects one or more cells in the microgroove or microtube;
imaging the one or more cells;
treating the one or more cells with one or more active agents; and
determining the effect of the one or more active agents on the one or more cells by detecting changes to the one or more cells in the microgrooves or microtubes.

35. The method of claim 34, wherein at least one of: the diameter of the target cell is between 1 to 100 μm in diameter; the one or more chambers are rectangular, circular, or triangular shaped; or a micropillar in the microtube is circular, rectangular or triangular shaped.

36. The method of claim 34, wherein at least one of: the target cell is a cancer cell, and the one or more active agents is an anti-neoplastic agent.

37. (canceled)

38. The method of claim 34, further comprising an imaging device, wherein the imaging device detects one or more cells in the microgroove or microtube.

39. The method of claim 34, wherein at least one of: the substrate is biocompatible or is a material coated to be biocompatible; or the substrate is selected from at least one of glass, silicon, polymer, plastic, metal, ceramic, semiconductor, or any combination thereof.

40. (canceled)

41. (canceled)

42. (canceled)

43. The method of claim 34, wherein the microgrooves or microtubes further comprise at least one of: a portion that narrows to a size that is less than a diameter of a target cell to trap the target cell; are on a first plane, and at least one of the input or output are on a second plane; are sized to hold one or more target cells; are between about 1 to 75 μm and a height depth of the microgroove is between about 1 to 100 μm; can hold one or more daughter cells from target cells; or are on a first plane, and at least one of the input or output are on a second plane, wherein the input, the output, or both are above or below the first plane.

44. (canceled)

45. (canceled)

46. The method of claim 34, wherein at least one of: the input comprises a first buffer exchange/feeding the input port or a fluid reservoir; or the output port comprises a reservoir or a site for aspiration of a fluid in the device.

47. (canceled)

48. The method of claim 34, wherein at least one of: a fluid drives the target cells or group of cells to enter the microgrooves or microtubes toward the fluid output or a fluid pressure drop is equally distributed among the microgrooves or microtubes.

49. (canceled)

50. (canceled)

51. (canceled)

52. The method of claim 34, wherein captured cells or groups of cells can be removed by reversing a flow, and replenished with new cells for further analysis.

53. The method of claim 34, further comprising capturing a cell or groups of cells, and adding reagents to stain specific components in the cell.
